(19) United States
(12) Patent Application Publication
(10) Pub. No.: US 2023/0398314 A1
(43) Pub. Date: Dec. 14, 2023

(54) INJECTION GLOVE

(71) Applicants: Sambhu N. Choudhury, Cincinnati, OH (US); Monika Rathnayake, Cincinnati, OH (US)

(72) Inventors: Sambhu N. Choudhury, Cincinnati, OH (US); Monika Rathnayake, Cincinnati, OH (US)

(21) Appl. No.: 18/333,246

(22) Filed: Jun. 12, 2023

Related U.S. Application Data
(60) Provisional application No. 63/351,553, filed on Jun. 13, 2022.

(51) Int. Cl. A61M 5/42; A61H 23/02; A61N 1/36; A61B 42/10

(52) U.S. Cl. CPC A61M 5/422; A61H 23/02; A61N 1/36021; A61B 42/10; A61H 2201/10; A61H 2201/165; A61H 2201/5002; A61H 2201/5038; A61H 2201/501; A61H 2201/5097

(57) ABSTRACT

An injection glove produces a variable vibration pattern that can be used to do injections with less pain. The glove is embedded with a communication module to a cellphone application that allows variation of the strength of the impulses, the digits of vibration, as well as various patterns of potential vibration.

### CROSS-REFERENCE TO RELATED APPLICATIONS

This application claims the benefit of priority under 35 U.S.C. § 119(e) to U.S. Patent Application Provisional Application Ser. No. 63/351,553, entitled “INJECTION GLOVE”, filed Jun. 13, 2022, the contents of which are hereby incorporated by reference in their entirety for any purpose.

### FIELD OF THE INVENTION

The present disclosure generally relates to therapeutic vibration delivery devices, and more particularly to garments having therapeutic vibration delivery devices.

### BACKGROUND OF THE INVENTION

Pain sensation is brought about through the use of local stimulation that results in a pain receptor creating an impulse in a pain nerve that then allows the person to feel pain. The nerves that control and pass the information of a painful stimulus also carries vibration and other information to the brain. Many studies have supported the gate control theory first described by Melzack et al. in 1965 to explain this physiological response. See, e.g., Melzack R, Wall PD: Pain Mechanisms: A new theory, Science 150:971-978, 1965.

The gate may be closed by stimulation of other nerve fibers using non-painful stimuli. Practical application of the gate control theory has led to widespread use of TENS for pain control. TENS stimulates large diameter sensory nerve fibers with mild repetitive electrical impulses via electrodes applied to the skin. Vibration is one stimulus that can decrease the impulses that represent pain and therefore make the original noxious stimulus less painful.

Vibration therapy is a safe and effective technique, which has long been used to alleviate pain. See, e.g., Vibration Therapy for Pain, Lancet. 1992. Jun. 20; 339(8808):1513-4.

Vibration stimuli around the procedure site may also act as a form of distraction, making it more difficult to identify exactly where and/or when a painful stimulus occurs.

Painful injections into and through the skin for immunizations, medication administration, blood sugar testing, phlebotomy, IV placement, and the like, are usually done without the use of a local or topical anesthetic.

Products to reduce the pain of injections and similar procedures do exist but have significant drawbacks and are not used often because of these limitations. Topical commercially available anesthetic creams such as EMLA (marketed by AstraZeneca) have a slow onset of action, requiring up to 90 minutes to be fully effective.

Ethyl chloride and other similar chemicals, long made available by corporations such as the Gebauer Company, act as a skin refrigerant to numb the skin prior to injections. Additional application of topical creams or tens unit or other electrical sensation like tens units have been used to decrease the pain response of an injection.

Another effective technique manually patting, vibrating, or stretching the skin around the site of injection just prior to a needle stick. This method also uses the gate theory mechanism by using stimuli to mask the pain of injection.

### BRIEF DESCRIPTION OF THE DRAWINGS

The various exemplary embodiments of the present invention, which will become more apparent as the description proceeds, are described in the following detailed description in conjunction with the accompanying drawings, in which:

FIG. 1 depicts a front view of an example injection glove assembly that produces a vibration pattern having a therapeutic or psychological benefit to a recipient, according to one or more embodiments;

FIG. 2 depicts a front view of the example injection glove assembly that is remotely controlled by a user device and while being used to produce a vibration pattern having a therapeutic or psychological benefit to a recipient of an injection, according to one or more embodiments; and

FIG. 3 depicts a functional block diagram of an example injection glove assembly 100 that performs the functionality of a controllable vibration pattern, according to one or more embodiments.

### DETAILED DESCRIPTION

According to aspects of the present disclosure, an injection glove minimizes pain associated with injections, and other similar procedures by providing a vibration stimulation to skin of a recipient. The present innovation comprises a glove apparatus with several vibratory components in multiple fingers. These fingers are typically the index, middle and thumb. Other areas of the glove support the battery and the computer chip that allows communication between the cellphone and the device. The battery can be charged inductively or with portal for wire placement to an outlet. The injection glove can provide topical analgesia during a procedure. The injection glove can include multiple vibratory elements placed within multiple glove digits.

The module on the user device can include a controller for controlling intensity, timing, and duration of the electrical vibration; and an input means for inputting a control signal to control the intensity and duration of the electrical stimuli and vibration. Different fingers can be chosen as the vibration tool. The controller may initially ramp up one or more and vibration, generate randomly timed bursts of one or more vibration. Alteration of the vibration stimulus can be done for the same patient with multiple injections or altered for different body parts based upon the needs for the area of the body.

The glove in one embodiment can be made of material that can be sterilized with sanitizer. Another embodiment will be to allow use of gloves latex, nitrile, to cover the glove and be disposed of after the procedure.

This may be accomplished by various means using rotational or oscillating vibration devices commonly found in cell phones and pagers, for example. One or more of these vibration devices may be placed in the multiple digits of the glove. Programmable micro controller circuitry may also be enclosed within the unit to control vibration stimuli. It may be pre-programmed to gradually increase both stimuli over a few seconds to a preset maximum level to prevent the initial sensation of surprise associated with a more sudden application of full stimulation. Variation of the stimulus can also be done manually on the phone. The optimum vibration for a patient may also be recorded on the cell phone application and reused on subsequent injections.

This micro controller may also be pre-programmed to deliver randomly timed bursts of vibration stimuli causing another form of distraction for the patient and making it more difficult to identify when the injection actually occurs.

Electrodes with the same basic features of the present invention may be shaped and sized to fit over specific body structures such as earlobes and fingers. The access window size and or shape may also be designed to accommodate different uses. Because of this, other medical applications may also benefit from this device including painful skin treatments such as laser therapy, skin biopsy, wart removal, splinter and hook removal, or any potentially painful procedure done at or near the skin surface. Painful cosmetic procedures such as ear and body piercing, tattooing, and hair removal may also be made more comfortable with the present innovation.

Furthermore, the described features, structures, or characteristics of the invention may be combined in any suitable manner in one or more embodiments. In the following description, numerous specific details are provided, such as examples of programming, software modules, user selections, network transactions, database queries, database structures, hardware modules, hardware circuits, hardware chips, etc., to provide a thorough understanding of embodiments of the invention. One skilled in the relevant art will recognize, however, that the invention can be practiced without one or more of the specific details, or with other methods, components, materials, and so forth.

By virtue of the present disclosure, the present innovation claims:

In one embodiment, the present invention provides for an apparatus for providing topical analgesia during a procedure, comprising a glove designed to have multiple vibratory elements in the digits and the thumb controlled with and connected with a cell phone app.

The stimulus of vibration can be varied per user to be in different fingers, different pulses, different patterns, and different intensities.

The fabric should allow for cleaning of the surface without contaminating the electrical device within the glove.

Additionally, the practitioner can choose to cover the glove with a latex or nitrile covering during the procedure and then exchange between patient encounters.

Programs can be saved for usage patterns per patient or per practitioner.

In one embodiment, the present invention provides for an injection glove produces a variable vibration pattern that can be used to do injections with less pain; wherein the glove is embedded with a communication module to connect to at least one user accessible electronic device (“user device”) with human interface functionality that allows variation of the strength of the impulses, the digits of vibration, as well as various patterns of potential vibration.

In another embodiment, the present invention provides for an injection glove further comprising multiple vibratory elements in the digits.

In another embodiment, the present invention provides for an injection glove wherein the multiple vibratory elements are controlled with and connected with the at least one user accessible electronic device.

In another embodiment, the present invention provides for an injection glove wherein the vibration can be varied per user to use variations selected from the group consisting of different fingers, pulses, patterns, and intensities.

In another embodiment, the present invention provides for an injection glove wherein the at least one user accessible electronic device comprises programs pre-sets allowing for usage patterns per patient or per practitioner.

In another embodiment, the present invention provides for an injection glove wherein the at least one user accessible electronic device is one of a mobile device, a smartphone, a personal digital assistant (PDA), a tablet, a wearable smart device, a phone, a cellular device, a cellphone, a mobile phone, a mobile terminal, an electronic tablet, and any other device configured to communicate using a wireless communication.

In another embodiment, the present invention provides for an injection glove wherein the at least one user accessible electronic device communicates by one of long-term evolution (LTE), global system for mobile communication (GSM), universal mobile telecommunications system (UMTS), enhanced data rates for GSM evolution (EDGE), code division multiple access (CDMA), and CDMA2000.

In another embodiment, the present invention provides for an injection glove wherein the glove is configured for reducing or eliminating the pain from injections or minor surgical procedures by the local application of vibrations about the injection or surgical site to block afferent pain fiber transmission.

In another embodiment, the present invention provides for an injection glove further comprising a plurality of projections extending outwardly from a surface of the glove adapted to be pressed against a subject for enhancing the stimulation of the subject during vibration of the glove.

In another embodiment, the present invention provides for an injection glove further comprising a current generating device configured to generate an electrical output Trans Epithelial Nerve Stimulating (TENS) current; and an array of electrodes electrically coupled to the current generating device and configured to be placed around an injection location on the skin of a patient.

In another embodiment, the present invention provides for an injection glove further comprising a glove housing and a pressure sensor coupled to the housing to detect contact between the injection glove and the subject's skin.

In another embodiment, the present invention provides for an injection glove further comprising a glove housing and a vibration mechanism coupled to an activation switch within the glove housing, that activates when pressed against a subject causes the injection glove to vibrate against the subject's skin during an injection to distract the patient from pain caused by the injection.

In another embodiment, the present invention provides for an injection glove further comprising a cooling mechanism coupled to the housing, that when activated, causes cooling of the injection glove before an injection to distract the subject from pain caused by the injection.

In another embodiment, the present invention provides for an injection glove further comprising a pressure sensor coupled to the housing to detect contact between the injection glove and the subject's skin.

In another embodiment, the present invention provides for an injection glove further comprising a temperature sensor coupled to the housing to detect a temperature of the injection glove.

In another embodiment, the present invention provides for methods of using the various injection gloves described herein.

In another embodiment, the present invention provides for an injection glove having a pressure tactile sensor comprising a microprocessor, an accelerometer component, a plurality of pressure sensing components, a gyroscope component and a power source. The accelerometer component is used to detect the acceleration of the glove and generate an acceleration signal, and transmit the acceleration signal to the microprocessor. Each pressure sensing component is located on the contact surface of each finger of the glove for detecting the pressure of each finger of the glove and generating a pressure value signal, and transmitting the pressure value signal to the microprocessor. The gyro element is used to detect the angle, posture and motion of the glove, generate an attitude signal, and transmit the attitude signal to the microprocessor. The power supply provides the power required by the glove.

In another embodiment, the present invention provides for an injection glove comprising well-known structures, materials, or operations are not shown or described in detail to avoid obscuring aspects of the invention. In another embodiment, the present invention provides for an injection glove comprising haptic feedback and may include a soft glove made of a flexible material. In another embodiment, the injection glove may utilize flexible, silicone-based smart textiles that contain an array of high-displacement pneumatic actuators and embedded microfluidic air channels. In another embodiment, the injection glove may include a lightweight force-feedback exoskeleton, which may be powered by microfluidic actuation. In another embodiment, the injection glove may incorporate motion tracking components. Motion tracking software may determine where a user's body is positioned in space to render convincing haptic interactions.

The present innovation can incorporate aspects of previous attempts to address pain and discomfort via use of a vibration pattern. The following references are hereby incorporated by reference in their entirety:

(1) Huttner, U.S. Pat. No. 6,902,554, describes a device which is pressed onto the skin and uses local pressure to elicit the gating mechanism as described above.

(2) Published U.S. Patent Application No. 2005/0149145, (Coulter, George Gary), discloses a pain reducing apparatus according to the preamble of claim 1, for use during therapeutic injection, (e.g., immunization), which has a current applying device coupled to current generating device for applying nerve stimulating current to electrodes placed around injection location.

(3) Published U.S. Patent Application No. 2004/0015188, (Coulter, George Gary), discloses a therapeutic injection or sampling device and process which comprises a mechanism for generating electrical output for a Trans Epithelial Nerve Stimulating current and mechanism for applying to patient's body part.

(4) European Patent No. 1699522, (COULTER, GEORGE GARY), discloses a hemorrhage reducing apparatus for use during therapeutic injection, which has electrodes placed around injection location on skin of a patient.

(5) Published U.S. Patent Application No. 2005/0177201, (Freeman, Gary A), discloses insertion of a probe element through the skin to a penetration depth for treatment, which involves moving the probe element along a penetration depth in a series of incremental movements. In the field of acupuncture, pre-treatment of the insertion area with electrical energy, often in the form of high frequency waveforms is typically used for transcutaneous electrical nerve stimulation (TENS), is employed to reduce the discomfort of insertion as well as provide optimal placement and treatment.

(6) Published U.S. Patent Application No. 2003/0187490, (Gliner, Bradford Evan), discloses an annular electrode for neural stimulation, which has an annular outer contact enclosing area, which is several times greater than neural cell structure area.

(7) Published U.S. Patent Application No. 2003/0181960, (Carter, John), discloses an electrotherapy apparatus for providing therapeutic electric current to a treatment site of a patient, which has a generator providing two pulsing electric alternating currents, a feed electrode, and a return electrode.

(8) U.S. Pat. No. 5,776,170 (MacDonald, Alexander John Ranald), discloses an Electrical stimulation analgesia apparatus for electrotherapy, which supplies electrical pulses with rapid rising and falling phases to electrodes on body surface to stimulate analgesia effects in central nervous system.

(9) U.S. Pat. No. 5,366,489 (Burgio, Paul A.), discloses an anesthesia electrode and applicator assembly for TENS, with active electrodes and return electrodes having a common carrier with a field of pressure sensitive adhesive for adhering electrode to the hand of practitioner or applicator.

(10) U.S. Pat. No. 6,516,226 (Bishay, Jon M), discloses a percutaneous electrical therapy system, which has an electrode housing, which supports and guides an electrode in a correct way during insertion of the electrode.

(11) U.S. Pat. No. 6,741,889 (Holcomb, Robert R), discloses an electromagnetic treatment device e.g., for pain and swelling which has an alternating polarity quadripolar array which generates a three-dimensional steep field gradient to alter stability of excitable membranes to treat ailments.

(12) Published U.S. Patent Application No. 2005/0089861, (Allen, John J), discloses lancing to obtain a sample of blood, which involves completely withdrawing the sharpened tip from incision, and drawing blood through the channel to the sensor. Sensor strip may be, for example, a glucose sensor strip which uses electrochemistry to measure the amount of glucose in a bodily fluid, such as, for example, blood or interstitial fluid.

(13) Published U.S. Patent Application No. 2002/0019652, (Da Silva, Luiz B), discloses a sterile bandage is combined with a TENS device for use in covering a wound and providing electrical stimulation to promote healing and block pain.

(14) U.S. Pat. No. 4,458,696 (Larimore, Franklin C), discloses a self-adhering TENS electrode extensible with the skin, comprising body-conformable conductive adhesive and connector layers eliminate dry-out problems.

(15) U.S. Pat. No. 6,871,099 (Whitehurst, Todd K), discloses a chronic pain e.g., migraine, treating method, involves providing operating power and stimulation parameters to stimulator to generate stimulation pulses based on parameters and delivering pulses to nerves and tissue.

(16) U.S. Pat. No. 5,423,874 (D'Alerta, Mario), discloses an electronic patch for applying pain reducing electrical energy to a body, which has an electronic circuit formed in a patch for generating and delivering electrical energy through afflicted region on patient's body.

(17) U.S. Pat. No. 5,904,712 (Axelgaard, Jens), discloses a transcutaneous medical electrode, which uses a grid of conductive arrays, each with selection of electrical connections to sections of arrays.

(18) U.S. Pat. No. 4,177,817 (Bevilacqua, Albert J.;), discloses a transcutaneous stimulation pulse electrode assembly, which has two electrolyte-filled chambers between adhesive coated surfaces and electric contacts.

(19) Published U.S. Patent Application No. 2002/0013602, (Huttner, James J.), discloses a method of controlling pain from surgical injections and minor medical procedures, which involves urging skin engaging surface of pressure member against skin of patient proximate the site.

(20) U.S. Pat. No. 4,289,136 (Rienzo, Sr., Donald D.), discloses a percutaneous pain alleviation system, which produces variable amplitude light-angled sawtooth pulses at its two electrodes, and has output current control.

(21) U.S. Pat. No. 4,989,605 (Rossen, Joel), discloses a pain treatment micro-current transcutaneous nerve stimulator, which uses a modulated monophasic sequence of bursts of DC carrier supplied to patient via electrodes.

(22) U.S. Pat. No. 6,907,299, (Han, Shu-Chang), discloses an electrode for transcutaneous electric nerve stimulator which has a conductive element made of carbon fiber, whose impedance is less than specified value.

(23) U.S. Pat. No. 6,904,324, (Bishay, Jon M.), discloses a percutaneous probe deploying apparatus to pierce the skin surface using electrodes for use in electrical nerve stimulation to treat pain in tissue.

(24) Published U.S. Patent Application No. 2003/0195599, (Bishay, Jon M.), discloses a percutaneous probe deploying apparatus to pierce the skin surface using electrodes for use in electrical nerve stimulation to treat pain in tissue.

(25) Published U.S. Patent Application No. 2006/0206164, (Gavronsky, Stas), discloses a percutaneous electrical nerve-stimulating device for electro-acupuncture, which has a needle/electrode holder including a linear electrode/needle guide channel, and pin electrode connecting needle/electrode to source of electric pulses.

(26) U.S. Pat. No. 4,784,142, (Liss, Saul;), discloses an electronic dental analgesia method using electrodes on the head and gums to pass an electric wave through patients' nerve system to suppress perceived pain.

(27) U.S. Pat. No. 3,620,209 (Kravitz, Harvey), discloses a reusable vibrating electrical device, which is strapped onto the arm of a patient in order to attenuate the pain of an injection by delivering vibration stimuli around the injection site.

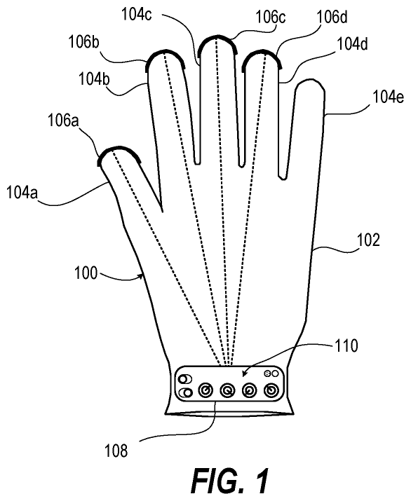

Turning to the Drawings, FIG. 1 depicts a front view of an example injection glove assembly 100 that produces a vibration pattern having a therapeutic or psychological benefit to a recipient. A glove 102 typically includes fingers 104a-104e. The glove 102 can be formed of a hypoallergenic and sterilized material. The glove 102 can be coated with an analgesic product. Aspects of the present innovation can also be applied to a particular finger cover or other harnesses or garments. One or more of fingertips of fingers 104a-104e include a respective electrical vibration actuator 106a-106d, such as fingers 104a-104d that exclude pinky finger 104e. A control module 108 is electrically connected to electrical vibration actuators 106a-106d. The control module 108 can include local user controls 110 to select a particular vibration pattern with programmed or adjusted intensity, frequency, duration, etc., for each electrical vibration actuator 106a-106d. The electrical vibration actuator 106a-106d can include vibratory motors or other technology that is be arranged in any practical arrangement such that the vibration pattern is transferred from the glove 102 to the recipient. First generation designs of haptic feedback use electromagnetic technologies such as vibratory motors, like a vibrating alert in a cell phone or a voice coil in a speaker, where a central mass is moved by an applied magnetic field. These electromagnetic motors typically operate at resonance and provide strong feedback but produce a limited range of sensations and typically vibrate the whole device, rather than an individual section. Second generation haptics offer touch-coordinate specific responses, allowing the haptic effects to be localized to the position on a device rather than the whole device. Second generation haptic actuator technologies include electroactive polymers, piezoelectric, electrostatic, and subsonic audio wave surface actuation. Third generation haptics deliver both touch-coordinate specific responses and customizable haptic effects. The customizable effects can be created using low latency control chips. A new technique that does not require actuators is called reverse-electrovibration. A weak current is sent from a device on the user through the object they are touching to the ground. The oscillating electric field around the skin on their fingertips creates a variable sensation of friction depending on the shape, frequency, and amplitude of the signal.

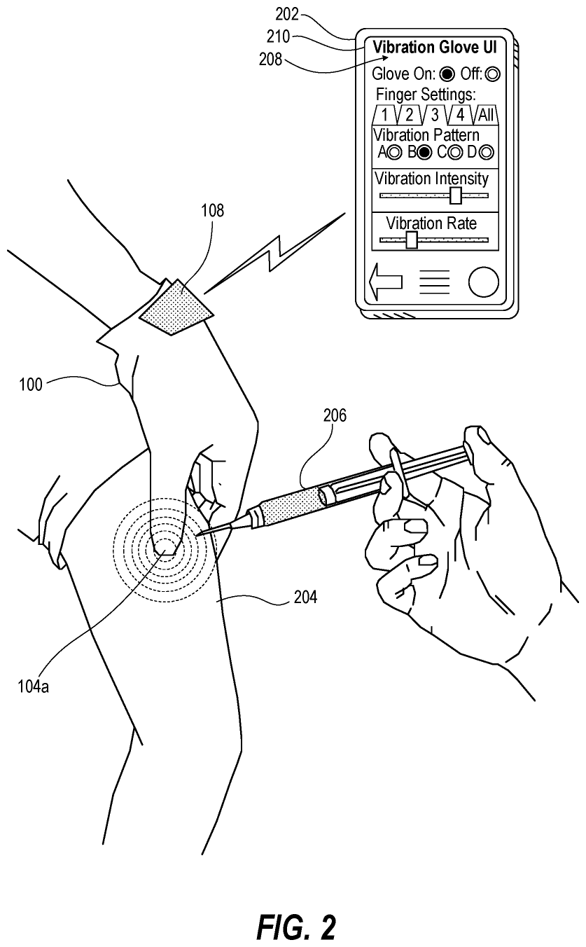

FIG. 2 depicts a front view of the example injection glove assembly 100 on a hand of a user 201 that is remotely controlled by at least one user device 202 and while being used to produce a vibration pattern, such as from finger 104a, having a therapeutic or psychological benefit to a recipient 204 of an injection by a syringe 206. The user device 202, such as a smartphone, presents a vibration user interface (UI) 208 that is presented on a touchscreen display 210. Examples of controls include selections for individual or collective fingers for a pre-defined vibration pattern that are appropriate for particular treatments, an adjustable vibration intensity, and adjustable vibration rate.

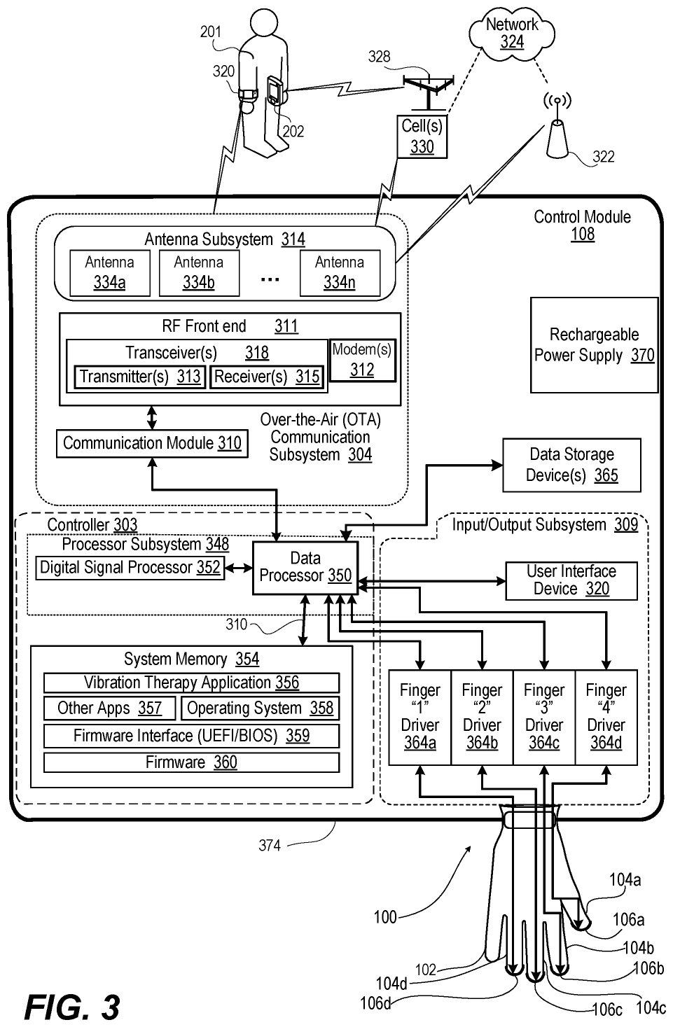

FIG. 3 is a functional block diagram illustrating example injection glove assembly 100 that performs the functionality of a controllable vibration pattern. The control module 108 of the injection glove assembly 100 includes over-the-air (OTA) communication subsystem 304 that communicates with user device 202. Injection glove assembly 100 provides computing and data storage functionality in support of OTA communication with user device 202. Injection glove assembly 100 also provides other functions with controller 303, data storage subsystem 308, and input/output (I/O) subsystem 309 that are communicatively coupled to each other via a system interlink 310.

OTA communication subsystem 304 includes communication module 310 that operates in baseband to encode data for transmission and decodes received data, according to a predetermined communication protocol. OTA communication subsystem 304 includes radio frequency (RF) front end 311 having one or more modem(s) 312. Modem(s) 312 modulate baseband encoded data from communication module 310 onto a carrier signal to provide a transmit signal that is amplified by transmitter(s) 313. Modem(s) 312 demodulates the received signal from node 322 detected by antenna subsystem 314. The received signal is amplified and filtered by receiver(s) 315, which demodulate received encoded data from a received carrier signal.

Controller 303 controls the OTA communication subsystem 304, user interface device 320, and other functions and/or operations of injection glove assembly 100. These functions and/or operations include, but are not limited to including, application data processing and signal processing. Injection glove assembly 100 may use hardware component equivalents for application data processing and signal processing. For example, injection glove assembly 100 may use special purpose hardware, dedicated processors, general purpose computers, microprocessor-based computers, micro-controllers, optical computers, analog computers, dedicated processors and/or dedicated hard wired logic. As utilized herein, the term “communicatively coupled” means that information signals are transmissible through various interconnections, including wired and/or wireless links, between the components. The interconnections between the components can be direct interconnections that include conductive transmission media or may be indirect interconnections that include one or more intermediate electrical components. Although certain direct interconnections (interlink 310) are illustrated in FIG. 3, it is to be understood that more, fewer, or different interconnections may be present in other embodiments.

In one or more embodiments, controller 303, via OTA communication subsystem 304, performs multiple types of OTA communication with external OTA communication system 306. OTA communication subsystem 304 can communicate with one or more personal access network (PAN) devices within external OTA communication system 306, such as smart watch 320 that is reached via Bluetooth connection. In one or more embodiments, OTA communication subsystem 304 communicates with one or more locally networked devices via a wireless local area network (WLAN) link provided by WLAN node 322. WLAN node 322 is in turn connected to wide area network 324, such as the Internet. In one or more embodiments, OTA communication subsystem 304 communicates with radio access network (RAN) 328 having respective base stations (BS s) or cells 330. RANs 328 are a part of a wireless wide area network (WWAN) that is connected to wide area network 324 and provides data services. In one or more embodiments, antenna subsystem 314 includes multiple antenna elements 334a— n that are individually tuned to selected RF bands to support different RF communication bands and protocols. Antenna elements 334a— n can be used in combination for multiple input multiple output (MIMO) operation for beam steering and spatial diversity.

Controller 303 includes processor subsystem 348, which executes program code to provide functionality of the injection glove assembly 100. Processor subsystem 348 includes one or more central processing units (CPUs) (“data processor”) 350. In one or more embodiments, processing subsystem 348 includes a digital signal processor (DSP) 352. Controller 303 includes system memory 354, which contains actively used program code and data. In one or more embodiments, system memory 354 includes therein a plurality of such program code and modules, including applications such as vibration pattern application 356 and other applications 357. System memory 354 can also include operating system (OS) 358, firmware interface 359 such as basic input/output system (BIOS) or Uniform Extensible Firmware Interface (UEFI), and platform firmware 360. These software and/or firmware modules have varying functionality when their corresponding program code is executed by processor subsystem 348 or secondary processing devices within injection glove assembly 100.

Data storage subsystem 308 provides nonvolatile storage accessible to controller 303. For example, data storage subsystem 308 can provide a large selection of other applications 357 that can be loaded into system memory 354. I/O subsystem 309 includes input and output devices such as a user interface device 320 and finger drivers 362a-364d that respectively drive electrical vibration actuator 106a-106d on fingers 104a-104d. Power for injection glove assembly 100 can be provided by a rechargeable power supply 370.

Some of the functional units described in this specification have been labeled as modules, or components, to more particularly emphasize their implementation independence. For example, a module may be implemented as a hardware circuit comprising custom very large scale integration (VLSI) circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in software for execution by various types of processors. An identified module of executable code may, for instance, comprise one or more physical or logical blocks of computer instructions that may, for instance, be organized as an object, procedure, or function. Nevertheless, the executables of an identified module need not be physically located together, but may comprise disparate instructions stored in different locations which, when joined logically together, comprise the module and achieve the stated purpose for the module.

Indeed, a module of executable code could be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices, and may exist, at least partially, merely as electronic signals on a system or network.

It must be noted that, as used in this specification and the appended claims, the singular forms “a,” “an” and “the” include plural referents unless the content clearly dictates otherwise. Thus, for example, reference to a “colorant agent” includes two or more such agents.

Unless defined otherwise, all technical and scientific terms used herein have the same meaning as commonly understood by one of ordinary skill in the art to which the invention pertains. Although a number of methods and materials similar or equivalent to those described herein can be used in the practice of the present invention, the preferred materials and methods are described herein.

As will be appreciated by one having ordinary skill in the art, the methods and compositions of the invention substantially reduce or eliminate the disadvantages and drawbacks associated with prior art methods and compositions.

It should be noted that, when employed in the present disclosure, the terms “comprises,” “comprising,” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

While it is apparent that the illustrative embodiments of the invention herein disclosed fulfill the objectives stated above, it will be appreciated that numerous modifications and other embodiments may be devised by one of ordinary skill in the art. Accordingly, it will be understood that the appended claims are intended to cover all such modifications and embodiments, which come within the spirit and scope of the present invention.

We claim:

1. An injection glove produces a variable vibration pattern that can be used to do injections with less pain; wherein the glove is embedded with a communication module to connect to at least one user accessible electronic device with human interface functionality that allows variation of the strength of the impulses, the digits of vibration, as well as various patterns of potential vibration.

2. The injection glove of claim 1, further comprising multiple vibratory elements in the digits.

3. The injection glove of claim 2, wherein the multiple vibratory elements are controlled with and connected with the at least one user accessible electronic device.

4. The injection glove of claim 2, wherein the vibration can be varied per user to use variations selected from the group consisting of different fingers, pulses, patterns, and intensities.

5. The injection glove of claim 3, wherein the at least one user accessible electronic device comprises programs pre-sets allowing for usage patterns per patient or per practitioner.

6. The injection glove of claim 3, wherein the at least one user accessible electronic device is one of a mobile device, a smartphone, a personal digital assistant (PDA), a tablet, a wearable smart device, a phone, a cellular device, a cellphone, a mobile phone, a mobile terminal, an electronic tablet, and any other device configured to communicate using a wireless communication.

7. The injection glove of claim 3, wherein the at least one user accessible electronic device communicates by one of long term evolution (LTE), global system for mobile communication (GSM), universal mobile telecommunications system (UMTS), enhanced data rates for GSM evolution (EDGE), code division multiple access (CDMA), and CDMA2000.

8. The injection glove of claim 3, wherein the glove is configured for reducing or eliminating the pain from injections or minor surgical procedures by the local application of vibrations about the injection or surgical site to block afferent pain fiber transmission.

9. The injection glove of claim 3, further comprising a plurality of projections extending outwardly from a surface of the glove adapted to be pressed against a subject for enhancing the stimulation of the subject during vibration of the glove.

10. The injection glove of claim 3, further comprising a current generating device configured to generate an electrical output Trans Epithelial Nerve Stimulating (TENS) current; and an array of electrodes electrically coupled to the current generating device and configured to be placed around an injection location on the skin of a patient.

11. The injection glove of claim 3, further comprising: a glove housing and a pressure sensor coupled to the housing to detect contact between the injection glove and the subject's skin.

12. The injection glove of claim 3, further comprising: a glove housing and a vibration mechanism coupled to an activation switch within the glove housing, that activates when pressed against a subject causes the injection glove to vibrate against the subject's skin during an injection to distract the patient from pain caused by the injection.

13. The injection glove of claim 3, further comprising: a cooling mechanism coupled to the housing, that when activated, causes cooling of the injection glove before an injection to distract the subject from pain caused by the injection.

14. The injection glove of claim 3, further comprising: a pressure sensor coupled to the housing to detect contact between the injection glove and the subject's skin.

15. The injection glove of claim 3, further comprising: a temperature sensor coupled to the housing to detect a temperature of the injection glove.
